(19) United States
(12) Patent Application Publication
(10) Pub. No.: US 2025/0352736 A1
(43) Pub. Date: Nov. 20, 2025

(54) Injector Having a Guard Assembly

(71) Applicant: Halozyme, Inc., San Diego, CA (US)

(72) Inventors: Kevin RUDA, Hopkins, MN (US); Kevin SWANSON, Plymouth, MN (US)

(73) Assignee: Halozyme, Inc., San Diego, CA (US)

(21) Appl. No.: 19/205,292

(22) Filed: May 12, 2025

Related U.S. Application Data
(60) Provisional application No. 63/649,060, filed on May 17, 2024.

(51) Int. Cl. A61M 5/32; A61M 5/46

(52) U.S. Cl. CPC A61M 5/326; A61M 5/3245; A61M 5/46; A61M 2207/00

(57) ABSTRACT

An injector includes a housing, a needle, and a needle shield removably coupled to the needle. A guard assembly is coupled to the housing and includes a guard holder coupled to the needle shield and having at least one engagement feature extending radially inward and a needle guard extension removably coupled to the guard holder. The needle guard extension is coupled to the guard holder and separated from the housing in an initial position, coupled to the guard holder and coupled to the housing in an engaged position, and separated from the guard holder and coupled to the housing in an active position. The guard holder couples to the needle shield to remove the needle shield from the needle when the needle shield extension is moved from the engaged position to the active position.

### CROSS-REFERENCE TO RELATED APPLICATIONS

This application claims the benefit of U.S. Provisional Patent Application No. 63/649,060 filed May 17, 2024 entitled “Injector Having a Guard Assembly”, which is incorporated by reference herein in its entirety.

### TECHNICAL FIELD

The present disclosure generally relates to injectors for injecting medicament into a patient and, in some embodiments, to an injector having a guard assembly.

### SUMMARY

In one embodiment, there is an injector including a housing having a proximal end and a distal end extending along a longitudinal axis extending therebetween, a needle extending from the distal end of the housing, a needle shield removably coupled to the needle and a guard assembly configured to couple to the distal end of the housing. In some embodiments, the guard assembly includes a guard holder having at least one engagement feature extending radially inward and configured to couple to the needle shield, and a needle guard extension removably coupled to the guard holder, the needle guard extension (i) coupled to the guard holder and separated from the distal end of the housing in an initial position, (ii) coupled to the guard holder and coupled to the distal end of the housing in an engaged position and (iii) separated from the guard holder and coupled to the distal end of the housing in an active position. In some embodiments, the guard holder couples to the needle shield to remove the needle shield from the needle when the needle guard extension is moved from the engaged position to the active position.

In some embodiments, the at least one engagement feature is deformable in response to the needle shield passing thereby when the needle guard extension is moved from the initial position to the engaged position. In some embodiments, the at least one engagement feature is deformable distally relative to the guard holder. In some embodiments, the at least one engagement feature includes a plurality of engagement features spaced circumferentially around the guard holder. In some embodiments, the at least one engagement feature is angled distally. In some embodiments, the at least one engagement feature prevents the needle shield from moving proximally relative to the guard holder when the needle guard extension is moved from the engaged position to the active position.

In some embodiments, the distal end of the housing includes threads. In some embodiments, the needle guard extension includes a knob extending radially therefrom, the knob configured to engage the threads of the housing. In some embodiments, the needle guard extension is threadedly coupled to the distal end of the housing in the engaged position.

In some embodiments, the injector includes a retractable needle guard extending at least partially from the distal end of the housing. In some embodiments, the needle guard extension is couplable with the retractable needle guard. In some embodiments, the needle guard extension is retractable relative to the housing between an extended position and a retracted position when the needle guard extension is in the active position. In some embodiments, the needle guard extension extends further from the distal end of the housing than the needle when the needle guard extension is in the extended position. In some embodiments, the needle is exposed when the needle guard extension is in the retracted position. In some embodiments, the needle extends a predetermined distance past a distal end of the needle guard extension when the needle guard extension is in the retracted position. In some embodiments, the predetermined distance is an insertion depth.

In some embodiments, the guard holder includes a sheath spaced radially apart from the guard holder. In some embodiments, the sheath has a diameter that is larger than the diameter of the guard holder and a diameter of the needle guard extension. In some embodiments, the needle guard extension is disposed between the guard holder and the sheath. In some embodiments, the guard holder is moveable relative to the needle guard extension along the longitudinal axis when the needle guard extension is moved from the engaged position to the active position. In some embodiments, the guard holder is prevented from rotating relative to the needle guard extension about the longitudinal axis when the needle guard extension is in the initial position and in the engaged position.

In another embodiment, there is an injector including a housing having a proximal end and a distal end extending along a longitudinal axis extending therebetween, a needle extending from the distal end of the housing, a needle shield removably coupled to the needle, and a guard assembly configured to couple to the distal end of the housing. In some embodiments, the guard assembly includes a guard holder having at least one engagement feature extending radially inward and configured to couple to the needle shield, and a needle guard extension removably coupled to the guard holder, the needle guard extension (i) coupled to the guard holder and separated from the distal end of the housing in an initial position, (ii) coupled to the guard holder and coupled to the distal end of the housing in an engaged position and (iii) separated from the guard holder and coupled to the distal end of the housing in an active position. In some embodiments, the at least one engagement feature is deformable in response to the needle shield passing thereby when the needle guard extension is moved from the initial position to the engaged position. In some embodiments, the guard holder couples to the needle shield to remove the needle shield from the needle when the needle guard extension is moved from the engaged position to the active position. In some embodiments, the needle guard extension is retractable relative to the housing between an extended position and a retracted position when the needle guard extension is in the active position.

In another embodiment, there is a method of coupling a needle guard to an injector having a housing having a proximal end and a distal end extending along a longitudinal axis extending therebetween, a needle extending from the distal end of the housing, and a needle shield removably coupled to the needle. In some embodiments, the method includes the steps of coupling a guard assembly to the distal end of the housing, the guard assembly having a guard holder including at least one engagement feature extending radially inward and configured to couple to the needle shield and a needle guard extension removably coupled to the guard holder and configured to couple to the distal end of the housing, separating the guard holder from the needle guard extension, thereby removing the needle shield from the needle, where the needle guard extension extends further from the distal end of the housing than the needle, and pressing the needle guard extension against an injection site of a subject, where the needle guard extension is retractable relative to the housing to expose the needle for delivery of a medicament to the subject.

In some embodiments, the at least one engagement feature deforms in response to the needle shield passing thereby when the guard assembly is coupled to the distal end of the housing. In some embodiments, the at least one engagement feature includes a plurality of engagement features spaced circumferentially around the guard holder. In some embodiments, the needle guard extension is threadedly coupled to the distal end of the housing, where the needle guard extension includes a knob extending radially therefrom and the housing includes a thread configured to receive the knob. In some embodiments, the needle extends a predetermined distance past a distal end of the needle guard extension when the needle guard extension is pressed against the injection site of a subject, and where the predetermined distance is an insertion depth.

### BRIEF DESCRIPTION OF THE DRAWINGS

The following detailed description of embodiments of the injector having a guard assembly, will be better understood when read in conjunction with the appended drawings of an exemplary embodiment. It should be understood, however, that the invention is not limited to the precise arrangements and instrumentalities shown.

In the drawings:

FIG. 1 is a perspective view of an injector having a guard assembly in accordance with an exemplary embodiment of the present invention;

FIG. 2 is an exploded view of the injector of FIG. 1;

FIG. 3 is a perspective view of the guard assembly of FIG. 1;

FIG. 4 is a partially transparent perspective view a guard holder of the guard assembly of FIG. 1;

FIG. 5 is a partially transparent perspective view a guard extension of the guard assembly of FIG. 1;

FIG. 6 is a perspective view of the injector of FIG. 1;

FIG. 7 is a cross-sectional view of the injector of FIG. 1;

FIG. 8 is a perspective view of a guard holder having a needle shield coupled thereto separated from the injector of FIG. 1;

FIG. 9 is a cross-sectional view of the guard holder having a needle shield coupled thereto of FIG. 8; and

FIG. 10 is a perspective view of the needle guard extension coupled to the injector of FIG. 1 in a retracted position.

### DETAILED DESCRIPTION

The present disclosure generally relates to injectors and, in some embodiments, to an injector having a guard assembly. Injectors are commonly used in the delivery of a volume of injectable fluid, such as medicament, to a patient for sub skin surface penetration (e.g., subcutaneous and intramuscular injections). Injectors can include a needle guard that is coupled to the housing and is moveable to cover the needle after use to provide protection against accidental needle sticks, which can lead to infections or injuries for healthcare workers and/or patients during handling, transportation, and disposal. Moreover, needle guards help safeguard the integrity of the needle itself before use, preventing it from being bent or damaged, which could compromise its ability to deliver the medicament during use.

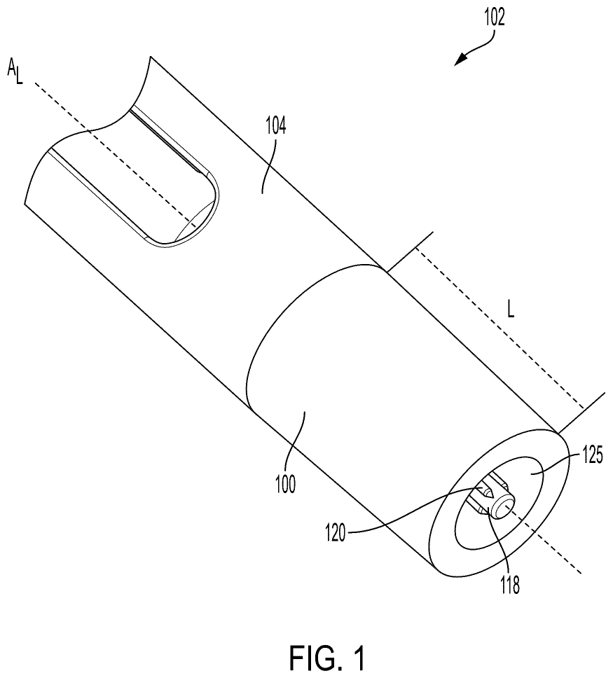

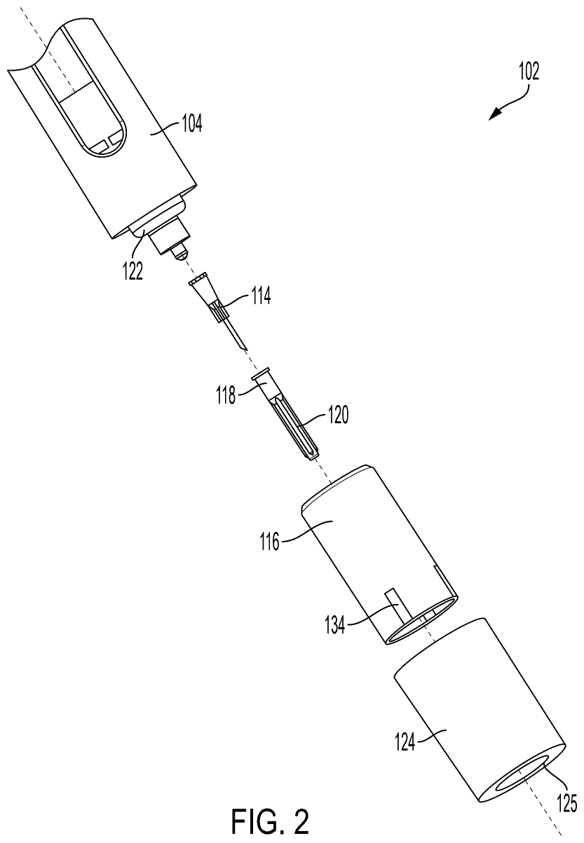

Referring to the drawings in detail, wherein like reference numerals indicate like elements throughout, there is shown in FIGS. 1-9, a guard assembly generally designated 100, in accordance with an exemplary embodiment of the present invention.

As shown in FIGS. 1, 2 and 5, an injector 102 may be configured to receive an accessory (e.g., the guard assembly 100) to cover a needle 114 prior to use of the injector 102. The guard assembly 100 may include a needle guard extension 116 that is configured to cover a needle 114 prior to use of the injector 102 and expose at least a portion of the needle 114 during use, as described in more detail below.

The injector 102 of FIGS. 1, 2, 6 and 10 may be an autoinjector containing a medicament and being configured to deliver the medicament to a subject. However, it should be understood that the injector 102 can be any injection apparatus with a needle 114 extending therefrom. The injector 102 may be configured to deliver a single-dose to the subject. The injector 102 may be configured for self-administration by a subject, or may be configured for administration by a user to the subject.

As shown in FIGS. 1, 2, 6-8 and 10, the injector 102 may include a housing 104. The housing 104 may have a proximal end and a distal end extending along a longitudinal axis A_{L} extending therebetween. The housing 104 may be configured to allow a user to grip or handle the injector 102. The housing 104 may be shaped to fit into a user's hand for single-handed function. The housing 104 may have a generally circular cross-section. In some embodiments, the housing 104 has a generally ovular cross-section to help position the injector 102 in the user's hand.

As shown in FIGS. 1, 2, 6, 7 and 10, the needle 114 may extend from the distal end of the housing 104. The needle 114 may be in fluid communication with a medicament container 122 disposed within the housing 104 to facilitate delivery of a medicament stored in the medicament container. The needle 114 may be a 25-gauge needle. In some embodiments, the needle 114 may be a 15-gauge needle, a 16-gauge needle, a 17-gauge needle, an 18-gauge needle, a 19-gauge needle, a 20-gauge needle, a 21-gauge needle, a 22-gauge needle, a 23-gauge needle, a 24-gauge needle, a 25-gauge needle, a 26-gauge needle, a 27-gauge needle, a 28-gauge needle, a 29-gauge needle, a 30-gauge needle, a 31-gauge needle, a 32-gauge needle, a 33-gauge needle, a 34-gauge needle, or a 35-gauge needle.

The needle 114 may have a length of approximately 0.1 inches, approximately 0.15 inches, approximately 0.2 inches, approximately 0.25 inches, approximately 0.3 inches, approximately 0.35 inches, approximately 0.4 inches, approximately 0.45 inches, approximately 0.5 inches, approximately 0.55 inches, approximately 0.6 inches, approximately 0.65 inches, approximately 0.7 inches, approximately 0.75 inches, approximately 0.8 inches, approximately 0.85 inches, approximately 0.9 inches, approximately 0.95 inches, approximately 1.0 inches, approximately 1.05 inches, approximately 1.1 inches, approximately 1.15 inches, and approximately 1.2 inches. The needle 114 may have a length of at least 0.1 inches, at least 0.15 inches, at least 0.2 inches, at least 0.25 inches, at least 0.3 inches, at least 0.35 inches, at least 0.4 inches, at least 0.45 inches, at least 0.5 inches, at least 0.55 inches, at least 0.6 inches, at least 0.65 inches, at least 0.7 inches, at least 0.75 inches, at least 0.8 inches, at least 0.85 inches, at least 0.9 inches, at least 0.95 inches, at least 1.0 inches, at least 1.05 inches, at least 1.1 inches, at least 1.15 inches, and at least 1.2 inches. The needle 114 may have a length between approximately 0.1 to 1.2 inches, between approximately 0.15 to 1.15 inches, between approximately 0.2 to 1.1 inches, between approximately 0.25 to 1.05 inches, between approximately 0.3 to 1.0 inches, between approximately 0.35 to 0.95 inches, between approximately 0.4 to 0.9 inches, between approximately 0.45 to 0.85 inches, between approximately 0.5 to 0.8 inches, between approximately 0.55 to 0.75 inches, between approximately 0.6 to 0.7 inches.

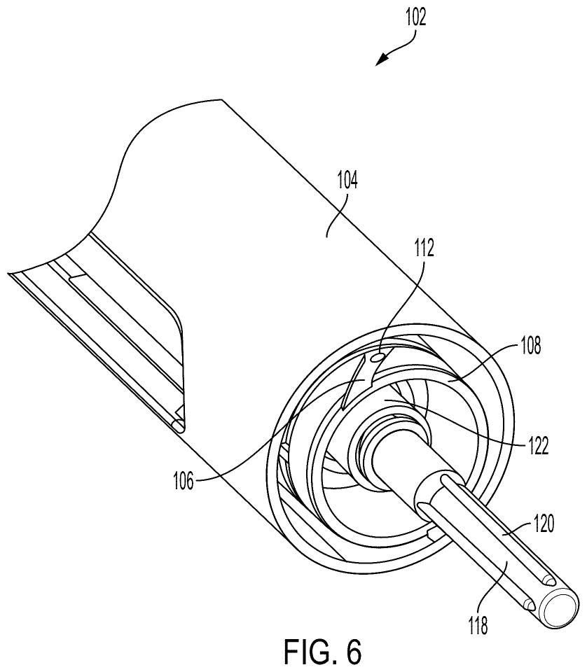

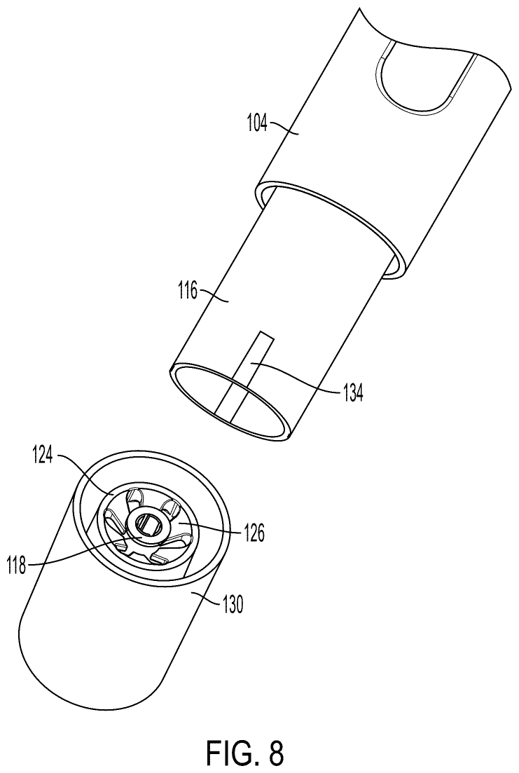

Referring to FIG. 6, before the guard assembly 100 is coupled to the injector 102, the needle 114 should be covered to prevent accidental insertion of the needle 114 or damage to the needle 114. A needle shield 118 may be removably coupled to the needle 114. The needle shield 118 may provide sharps protection and prevent unintentional needle insertions. The needle shield 118 may include one or more ribs 120 allowing a user to handle and rotate the needle 114 to couple the needle 114 to the medicament container 122 prior to use. The needle shield 118 may be removed from the needle, as shown in FIGS. 8-10 to expose the needle 114 for use, as described in more detail below.

As shown in FIG. 6, the injector 102 may be configured to receive the guard assembly 100 at the distal end. The distal end of the housing 104 may include a thread 106 defined thereon. The housing 104 may further include a needle guard 108 extending at least partially from the distal end of the housing 104. The needle guard 108 may be a retractable needle guard. At least a portion of the needle guard 108 may translate proximally into the housing in response to a proximal force acting thereon. In some embodiments, the entire needle guard 108 translates proximally into the housing 104 in response to a proximal force acting thereon. In some embodiments, the needle guard 108 is configured to receive guard assembly 100 at the distal end and the thread 106 is defined on the needle guard 108. The needle guard 108 may be urged distally relative to the housing 104 by a biasing element (not shown) to prevent the needle guard 108 from accidentally moving proximally relative to the housing 104 when the guard assembly 100 is being coupled thereto. The biasing element may be a coil spring, for example.

As shown in FIG. 6, the injector 102 may receive the guard assembly 100 at the distal end by introducing a feature of the guard assembly 100 to the thread 106 of the housing 104. The thread 106 may be an indent shaped and sized to receive a protruding component (e.g., knob 128 of the needle guard extension 116) of the guard assembly 100 to facilitate coupling of the guard assembly 100 to the injector 102. In some embodiments, the housing 104 includes the male component that is shaped and sized to be received in a female component of the guard assembly 100. The thread 106 may be defined on the distal end of the housing to facilitate engagement with the guard assembly 100. The thread 106 may be angled such that rotation of the guard assembly 100 relative to the injector 102 couples or decouples the guard assembly 100. For example, rotation of the guard assembly 100 in a first direction about the longitudinal axis A_{L} may couple the guard assembly 100 to the injector 102 and rotation of the guard assembly 100 in a second direction about the longitudinal axis A_{L} may decouple the guard assembly 100 from the injector 102. The first direction may be clockwise, and the second direction may be counterclockwise. In some embodiments, the first direction is counterclockwise and the second direction is clockwise.

The needle guard extension 116 may move proximally relative to the longitudinal axis A_{L} as the guard assembly 100 is coupled to the housing 104 as the knob 128 of the needle guard extension 116 is received in the thread 106. As the needle guard extension 116 is coupled to the housing 104, the guard holder 124 may be prevented from moving proximally relative to the longitudinal axis A_{L} by the distal end of the housing 104. In some embodiments, the guard assembly 100 is coupled to the housing 104 when the guard holder 124 engages the distal end of the housing 104. In some embodiments, the guard assembly 100 and the housing 104 are coupled together by at least one of a thread, friction fit, interference fit, snap fit, press fit and an adhesive.

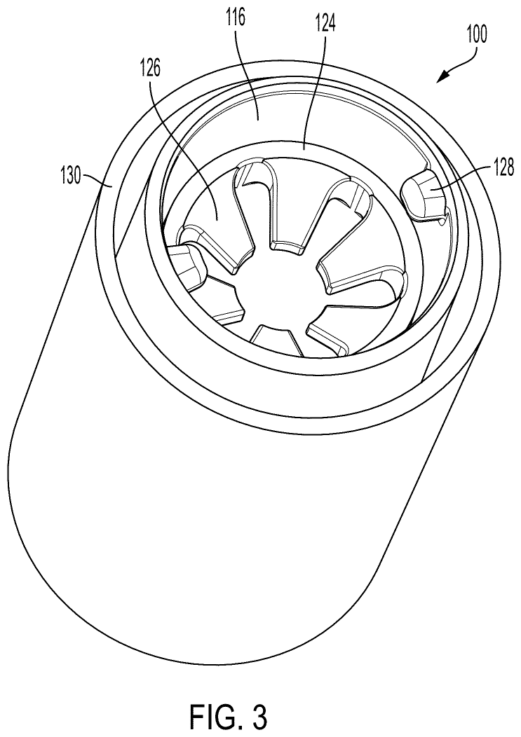

As shown in FIGS. 3-5, the guard assembly 100 may include the needle guard extension 116 and a guard holder 124. The needle guard extension 116 and the guard holder 124 may be generally cylindrical members. The guard holder 124 may have a diameter that is less than a diameter of the needle guard extension 116 to allow the guard holder 124 to be at least partially disposed within the needle guard extension 116. The guard holder 124 and the needle guard extension 116 may be coupled to one another by a friction fit. In some embodiments, the guard holder 124 and the needle guard extension 116 are coupled together by at least one of friction fit, interference fit, snap fit, press fit, a releasable adhesive, or a thread. In some embodiments, the guard holder 124 may have a diameter that is larger than the needle guard extension 116 to allow the needle guard extension 116 to be disposed at least partially therein.

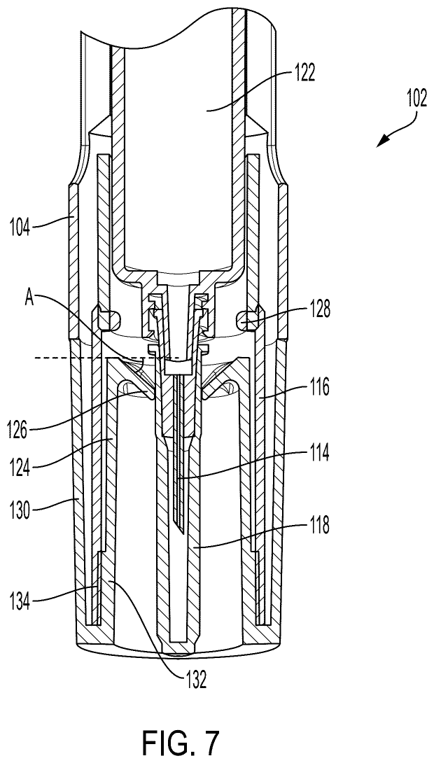

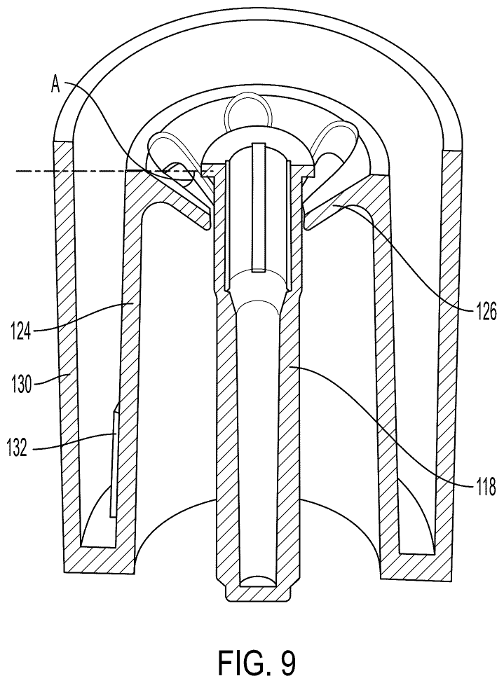

As shown in FIGS. 1, 4, 7 and 9, the guard assembly 100 may have an open distal end 125. The open distal end 125 of the guard assembly 100 may be shaped to allow at least a portion of the needle shield 118 to pass therethrough when the guard assembly 100 is coupled to the injector 102. The open distal end 125 may have a diameter that is larger than a diameter of the needle shield. The open distal end 125 may enable use of a needle 114 and needle shield 118 that extend from the housing 104 a distance that is longer than a length (L) of the guard assembly 100. As shown in FIGS. 7 and 9, the distance that the needle 114 and needle shield 118 extend from the housing 104 may be substantially similar to the length (L) of the guard assembly 100. In some embodiments, the distance that the needle 114 and needle shield 118 extend from the housing 104 is less than the length (L) of the guard assembly 100. In some embodiments, the guard assembly 100 has a closed distal end.

The needle guard extension 116 may enable the injector 102 to accommodate various needle 114 lengths required for different medical applications and may enable a user to swap out the needle guard extension 116 for different types or sizes of needles 114. This may provide healthcare providers or patients the ability to customize drug delivery protocols without obtaining separate equipment. The needle guard extension 116 may, therefore, ensure protection against needlestick injuries and safeguard the integrity of the needle during storage and use. Moreover, by coupling the needle guard extension 116 with or following the needle 114, the user is able to couple the needle 114 to the injector 102 before use without visual interference from a protruding needle guard. The user may also be able to visually inspect and confirm the volume of the medicament prior to an injection without obstruction from a retracted needle guard. This added visibility may enhance safety measures by ensuring proper assembly and alignment of components.

The needle guard extension 116 may be removably coupled to the guard holder 124 and may be configured to separate from the guard holder 124 in response to an opposite pulling force being applied to each of the guard holder 124 and the needle guard extension 116. The needle guard extension 116 may be configured to couple to the housing 104 and the guard holder 124 may be separated from the needle guard extension 116 by a user prior to use of the injector 102. The guard holder 124 and the needle guard extension 116 may be provided as part of a kit. The guard assembly 100 may be provided as part of a kit. The kit may include, for example, any or all of the guard assembly 100, the guard holder 124, the needle guard extension 116, the injector 102, the needle 114 and the needle shield 118.

The needle guard extension 116 may be moveable from an initial position prior to the guard assembly 100 being coupled to the housing 104, to an engaged position when the guard assembly 100 is coupled to the housing 104, to an active position in preparation for use of the housing 104. As shown in FIG. 3, the needle guard extension 116 may be coupled to the guard holder 124 and separated from the housing 104 in the initial position. The needle guard extension 116 may be in the initial position until the guard assembly 100 is coupled to the injector 102. As shown in FIGS. 1 and 7, the needle guard extension 116 may be coupled to the guard holder 124 and coupled to the housing 104 in the engaged position. The needle guard extension 116 may be in the engaged position when the guard assembly 100 is coupled to the injector 102. As shown in FIG. 8, the needle guard extension 116 may be separated from the guard holder 124 and coupled to the housing 104 in the active position. The needle guard extension 116 may be in the engaged position after the guard assembly 100 has been coupled to the injector 102 and the guard holder 124 is separated from the needle guard extension 116.

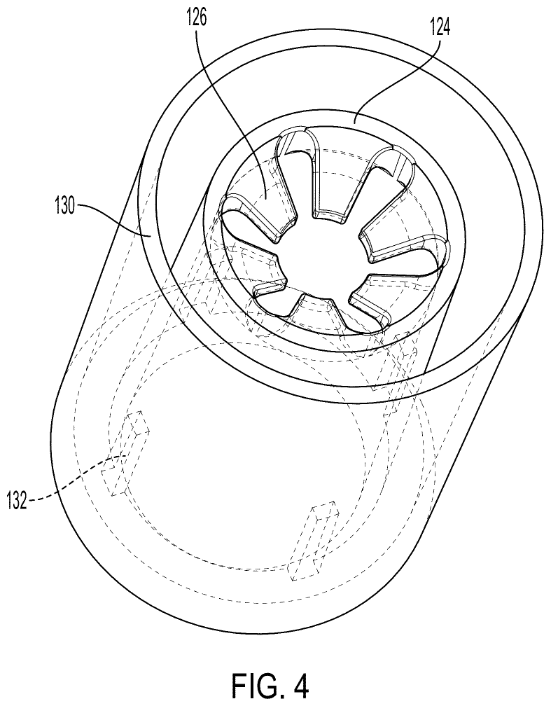

As shown in FIGS. 3-4, the guard holder 124 may include a sheath 130 spaced radially apart from the guard holder 124. The sheath 130 may have a length that is substantially similar to a length of the guard holder 124 and may be coupled to the guard holder 124 at one end. The sheath 130 and the guard holder 124 may be coupled at the distal end. As shown in FIG. 9, the guard holder 124 and the sheath 130 may form a u-shaped cross section when coupled at the distal end. The sheath 130 and the guard holder 124 may be substantially parallel to one another. In some embodiments, the sheath 130 and the guard holder 124 are closer at a distal end than at a proximal end. The sheath 130 may be a generally cylindrical member and may have a diameter that is larger than the diameter of the guard holder 124 and the diameter of the needle guard extension 116. The needle guard extension 116 may be at least partially disposed between the guard holder 124 and the sheath 130 when the needle guard extension 116 is in the initial position and in the engaged position. The sheath 130 and the needle guard extension 116 may be coupled to one another by a friction fit. In some embodiments, the sheath 130 and the needle guard extension 116 are coupled together by at least one of friction fit, interference fit, snap fit, press fit, a releasable adhesive, or a thread. In some embodiments, the needle guard extension 116 is coupled to the guard holder 124 and the sheath 130.

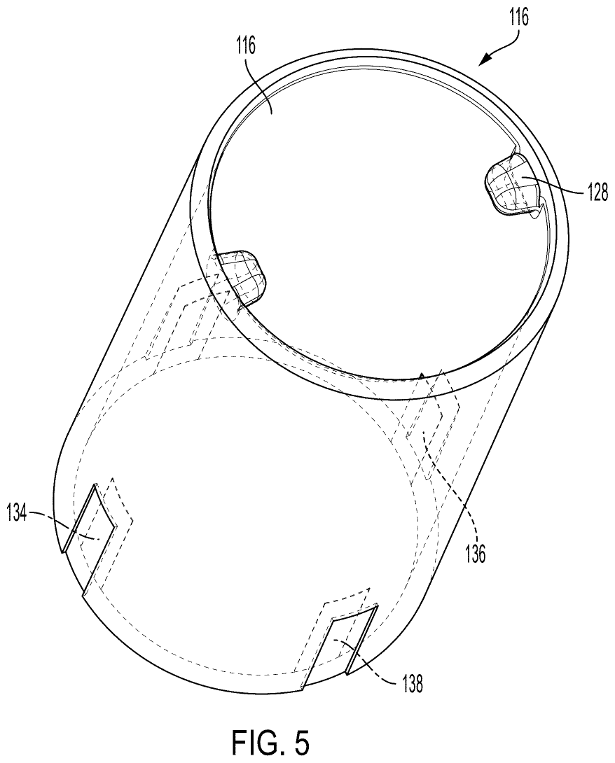

The guard holder 124 may be prevented from rotating relative to the needle guard extension 116 about the longitudinal axis A_{L} when the needle guard extension 116 is in the initial position and in the engaged position. As shown in FIGS. 4-5, the guard holder 124 and the needle guard extension 116 may include features defined thereon configured to engage to prevent rotation of the guard holder 124 relative to the needle guard extension 116. Preventing rotation of the guard holder 124 relative to the needle guard extension 116 may improve the ease of coupling the needle guard extension 116 to the housing 104 while the user contacts the sheath 130.

As shown in FIG. 4, the guard holder 124 may include a holder protrusion 132 defined thereon. The holder protrusion 132 may be a generally rectangular member and protrude outwardly from the guard holder 124 and may extend along a plane that is generally parallel to the longitudinal axis A_{L} from a distal end of the guard holder 124 at least partially to a proximal end. In some embodiments, the holder protrusion 132 extends from a location proximate the distal end of the guard holder 124 at least partially to the proximal end. The guard holder 124 may include a plurality of holder protrusions 132 spaced apart around a circumference of the guard holder 124. In some embodiments, the holder protrusion 132 protrudes inwardly from the sheath 130 and extends along the sheath 130. In some embodiments, the guard holder 124 and the sheath 130 include a holder protrusion 132.

The holder protrusion 132 may be shaped and sized to be received in an extension recess 134 of the needle guard extension 116. As shown in FIG. 5, the needle guard extension 116 may include an extension recess 134 defined thereon. The extension recess 134 may be a generally rectangular indentation that extends into the needle guard extension 116 and may extend along a plane that is generally parallel to the longitudinal axis A_{L} from a distal end of the needle guard extension 116 at least partially to a proximal end thereof. The needle guard extension 116 may include an interior extension recess 136 defined on an interior surface and an exterior extension recess 138 defined on an exterior surface thereof. The needle guard extension 116 may include both an interior extension recess 136 and an exterior extension recess 138 to accommodate a holder protrusion 132 that protrudes from the guard holder 124 or the sheath 130.

To facilitate coupling with the housing 104, the needle guard extension 116 may include a coupling feature. As shown in FIGS. 3 and 5, the needle guard extension 116 may include a knob 128 extending radially therefrom. The knob 128 may extend along a plane that is generally perpendicular to the longitudinal axis A_{L} at a location proximate the distal end of the needle guard extension 116. The knob 128 may be configured to engage the thread 106. The knob 128 may be shaped and sized to be at least partially disposed within the thread 106 when the needle guard extension 116 is in the engaged position and in the active position.

As shown in FIG. 6, the thread 106 may include a thread notch 112 defined thereon. The thread notch 112 may protrude from a surface of the thread 106 at a proximal-most region of the thread. In some embodiments, the thread notch 112 is located proximate the proximal-most region of the thread. The thread notch 112 may be configured to indicate to a user that the needle guard extension 116 is coupled to the housing 104 when the knob 128 drags over the thread notch 112. The thread notch 112 may generate an audible indication, such as a click. The thread notch 112 may cause resistance to rotation of the needle guard extension 116 relative to the housing 104 such that when the resistance is overcome the user would understand that the needle guard extension 116 is in the engaged position. In some embodiments, the needle guard extension 116 and the housing 104 may include an indicia (e.g., a sticker, etching, marking, etc.) that aligns when the needle guard extension 116 is in the engaged position.

As shown in FIG. 7, when the needle guard extension 116 is coupled to the housing 104, the guard holder 124 may be configured to couple to the needle shield 118. The guard holder 124 may have at least one engagement feature 126 extending radially inward therefrom. As shown in FIG. 9, the at least one engagement feature 126 may extend from the proximal end of the guard holder 124 and may be angled distally at an angle A.

Angle A may be approximately 25 degrees, approximately 30 degrees, approximately 35 degrees, approximately 40 degrees, approximately 45 degrees, approximately 50 degrees, approximately 55 degrees, approximately 60 degrees, and approximately 65 degrees. Angle A may be at least 25 degrees, at least 30 degrees, at least 35 degrees, at least 40 degrees, at least 45 degrees, at least 50 degrees, at least 55 degrees, at least 60 degrees, and at least 65 degrees. Angle A may be between approximately 25 to 65 degrees, between approximately 30 to 60 degrees, between approximately 35 to 55 degrees, and between approximately 40 to 50 degrees.

In some embodiments, the at least one engagement feature 126 extends from a location proximate the proximal end of the guard holder 124. In some embodiments, the at least one engagement feature extends along a plane that is generally perpendicular to the longitudinal axis A_{L}. The at least one engagement feature 126 may be deformable in response to the needle shield 118 passing thereby when the needle guard extension 116 is moved from the initial position to the engaged position. The at least one engagement feature 126 may be deformable distally relative to the guard holder 124 when the needle guard extension 116 is moved from the initial position to the engaged position.

The at least one engagement feature 126 may be configured to couple the guard holder 124 to the needle shield 118. The guard holder 124 may include a plurality of engagement features 126 spaced circumferentially around the guard holder 124. While six engagement features 126 are shown in FIGS. 3-4, it should be understood that in some embodiments the guard holder includes 2 engagement features 126, 3 engagement features 126, 4 engagement features 126, 5 engagement features 126, 6 engagement features 126, 7 engagement features 126, 8 engagement features 126, 9 engagement features 126, or 10 engagement features 126. In some embodiments, the at least one engagement feature is a circumferential member extending at least partially around the circumference of the guard holder with an aperture or septum extending therethrough sized and shaped to couple to the needle shield 118 when the guard assembly 100 is coupled to the housing 104.

As shown in FIGS. 8-9, the guard holder 124 may be separated from the needle guard extension 116 along the longitudinal axis A_{L} when the needle guard extension 116 is moved from the engaged position to the active position. The at least one engagement feature 126 may prevent the needle shield 118 from moving proximally relative to the guard holder 124 thereby coupling the needle shield 118 to the guard holder 124. The needle shield 118 may be removed from the needle 114 when the needle guard extension 116 is moved from the engaged position to the active position. The guard holder 124 and the needle shield 118 may be conveniently disposed of after separation from the needle guard extension 116 and needle 114, respectively. In some embodiments, the guard holder 124 is reusable. In some embodiments, the guard holder 124 and the needle shield 118 are reusable.

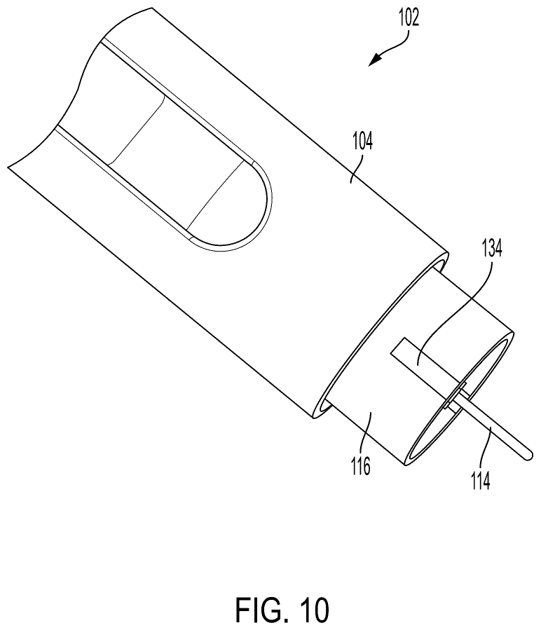

When the needle guard extension 116 is in the active position, the injector 102 may be used by the user. The needle guard extension 116 may be retractable relative to the housing 104 between an extended position (FIG. 8) and a retracted position (FIG. 10) when the needle guard extension 116 is in the active position. The needle guard extension 116 may extend a first distance from the housing in the extended position. The first distance may be determined based on the needle 114 length to prevent access to the needle 114 when the needle guard extension 116 is in the active position. The needle 114 may extend a second distance from the housing. The first distance may be greater than the second distance. The needle guard extension 116 may extend further from the distal end of the housing 104 than the needle 114 when the needle guard extension 116 is in the extended position.

The first distance may be approximately 0.5 inches, approximately 0.6 inches, approximately 0.7 inches, approximately 0.8 inches, approximately 0.9 inches, approximately 1.0 inches, approximately 1.1 inches, approximately 1.2 inches, approximately 1.3 inches, approximately 1.4 inches, or approximately 1.5 inches. The first distance may be at least 0.5 inches, at least 0.6 inches, at least 0.7 inches, at least 0.8 inches, at least 0.9 inches, at least 1.0 inches, at least 1.1 inches, at least 1.2 inches, at least 1.3 inches, at least 1.4 inches, or at least 1.5 inches. The first distance may be between approximately 0.5 to 1.5 inches, approximately 0.6 to 1.4 inches, approximately 0.7 to 1.3 inches, approximately 0.8 to 1.2 inches, or approximately 0.9 to 1.1 inches.

The second distance may be approximately 0.5 inches, approximately 0.6 inches, approximately 0.7 inches, approximately 0.8 inches, approximately 0.9 inches, approximately 1.0 inches, approximately 1.1 inches, approximately 1.2 inches, approximately 1.3 inches, approximately 1.4 inches, or approximately 1.5 inches. The second distance may be at least 0.5 inches, at least 0.6 inches, at least 0.7 inches, at least 0.8 inches, at least 0.9 inches, at least 1.0 inches, at least 1.1 inches, at least 1.2 inches, at least 1.3 inches, at least 1.4 inches, or at least 1.5 inches. The second distance may be between approximately 0.5 to 1.5 inches, approximately 0.6 to 1.4 inches, approximately 0.7 to 1.3 inches, approximately 0.8 to 1.2 inches, or approximately 0.9 to 1.1 inches.

The needle 114 may be exposed when the needle guard extension 116 is in the retracted position. The needle 114 may extend a predetermined distance past a distal end of the needle guard extension 116 when the needle guard extension 116 is in the retracted position. The predetermined distance may be an insertion depth of the needle 114.

The predetermined distance may be approximately 0.25 inches, approximately 0.3 inches, approximately 0.35 inches, approximately 0.4 inches, approximately 0.45 inches, approximately 0.5 inches, approximately 0.55 inches, approximately 0.6 inches, approximately 0.65 inches, approximately 0.7 inches, approximately 0.75 inches, approximately 0.8 inches, approximately 0.85 inches, approximately 0.9 inches, approximately 0.95 inches, or approximately 1.0 inches. The predetermined distance may be at least 0.25 inches, at least 0.3 inches, at least 0.35 inches, at least 0.4 inches, at least 0.45 inches, at least 0.5 inches, at least 0.55 inches, at least 0.6 inches, at least 0.65 inches, at least 0.7 inches, at least 0.75 inches, at least 0.8 inches, at least 0.85 inches, at least 0.9 inches, at least 0.95 inches, or at least 1.0 inches. The predetermined distance may be between approximately 0.25 inches and 1.0 inches, between approximately 0.3 inches and 0.95 inches, between approximately 0.35 inches and 0.9 inches, between approximately 0.4 inches and 0.85 inches, between approximately 0.45 inches and 0.8 inches, between approximately 0.5 inches and 0.75 inches, between approximately 0.55 inches and 0.7 inches, or between approximately 0.6 inches and 0.65 inches.

Referring to FIGS. 1-10, a method of coupling a needle guard extension 116 to an injector 102 may be provided herein. As shown in FIGS. 1, 2, 6-8 and 10, the injector 102 may have a housing 104 having proximal end and a distal end extending along a longitudinal axis A_{L}. A needle 114 may extend from the distal end of the housing 104, and a needle shield 118 may be removably coupled to the needle 114. The user may couple the needle 114 to a medicament container 122 disposed within the housing 104. The user coupling the needle 114 to the medicament container 122 prior to use may provide improved sterility of the components by allowing them to remain in a sealed and/or sterilized environment until use. The user may remove a plug from the distal end of the medicament container to enable the needle 114 to be coupled thereto. In some embodiments, the user may receive the injector 102 with the needle 114 coupled thereto. The plug may be a luer plug.

As shown in FIG. 1, the method may include coupling a guard assembly 100 to the distal end of the housing 104. In some embodiments, the guard assembly 100 may have the needle 114 coupled thereto. The user may couple the needle 114 to the medicament container 122 and the guard assembly 100 to the housing 104 simultaneously. The guard assembly 100 may have a guard holder 124 including at least one engagement feature 126 extending radially inward and configured to couple to the needle shield 118. The guard assembly 100 may have a needle guard extension 116 removably coupled to the guard holder 124 and configured to couple to the distal end of the housing 104. The guard holder 124 may be separably coupled to the needle guard extension 116.

The needle guard extension 116 may be threadedly coupled to the distal end of the housing 104, where the needle guard extension 116 may include a knob 128 extending radially therefrom and the housing 104 may include a thread 106 configured to receive the knob 128. The at least one engagement feature 126 may deform in response to the needle shield 118 passing thereby when the guard assembly 100 is coupled to the distal end of the housing 104. The at least one engagement feature 126 may include a plurality of engagement features 126 spaced circumferentially around the guard holder 124.

As shown in FIG. 8, the method may include separating the guard holder 124 from the needle guard extension 116, thereby removing the needle shield 118 from the needle 114. The needle guard extension 116 may extend further from the distal end of the housing 104 than the needle 114.

As shown in FIG. 10, the needle guard extension 116 may be retractable relative to the housing 104 to expose the needle 114 for delivery of a medicament to the subject. The method may include pressing the needle guard extension 116 against an injection site of a subject. Retraction of the needle guard extension 116 may trigger the injector 102. The needle 114 may extend a predetermined distance past a distal end of the needle guard extension 116 when the needle guard extension 116 is pressed against the injection site of a subject. The predetermined distance may be an insertion depth. The method may include removing the needle 114 from the subject after the medicament has been ejected therethrough. A user may dispose of the injector 102 after use.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

What is claimed is:

1. An injector comprising:
a housing having a proximal end and a distal end extending along a longitudinal axis extending therebetween;
a needle extending from the distal end of the housing;
a needle shield removably coupled to the needle; and
a guard assembly configured to couple to the distal end of the housing, the guard assembly including:
a guard holder having at least one engagement feature extending radially inward and configured to couple to the needle shield; and
a needle guard extension removably coupled to the guard holder, the needle guard extension (i) coupled to the guard holder and separated from the distal end of the housing in an initial position, (ii) coupled to the guard holder and coupled to the distal end of the housing in an engaged position and (iii) separated from the guard holder and coupled to the distal end of the housing in an active position,
wherein the guard holder couples to the needle shield to remove the needle shield from the needle when the needle guard extension is moved from the engaged position to the active position.

2. The injector of claim 1, wherein the at least one engagement feature is deformable in response to the needle shield passing thereby when the needle guard extension is moved from the initial position to the engaged position.

3. The injector of claim 2, wherein the at least one engagement feature is deformable distally relative to the guard holder.

4. The injector of claim 3, wherein the at least one engagement feature includes a plurality of engagement features spaced circumferentially around the guard holder.

5. The injector of claim 3, wherein the at least one engagement feature is angled distally.

6. The injector of claim 2, wherein the at least one engagement feature prevents the needle shield from moving proximally relative to the guard holder when the needle guard extension is moved from the engaged position to the active position.

7. The injector of claim 1, wherein the distal end of the housing includes threads.

8. The injector of claim 7, wherein the needle guard extension includes a knob extending radially therefrom, the knob configured to engage the threads of the housing.

9. The injector of claim 8, wherein the needle guard extension is threadedly coupled to the distal end of the housing in the engaged position.

10. The injector of claim 1 further comprising:
a retractable needle guard extending at least partially from the distal end of the housing.

11. The injector of claim 10, wherein the needle guard extension is couplable with the retractable needle guard.

12. The injector of claim 1, wherein the needle guard extension is retractable relative to the housing between an extended position and a retracted position when the needle guard extension is in the active position.

13. The injector of claim 12, wherein the needle guard extension extends further from the distal end of the housing than the needle when the needle guard extension is in the extended position.

14. The injector of claim 12, wherein the needle is exposed when the needle guard extension is in the retracted position.

15. The injector of claim 12, wherein the needle extends a predetermined distance past a distal end of the needle guard extension when the needle guard extension is in the retracted position, and
wherein the predetermined distance is an insertion depth.

16. The injector of claim 1, wherein the guard holder includes a sheath spaced radially apart from the guard holder.

17. The injector of claim 16, wherein the sheath has a diameter that is larger than the diameter of the guard holder and a diameter of the needle guard extension.

18. The injector of claim 16, wherein the needle guard extension is disposed between the guard holder and the sheath.

19. The injector of claim 1, wherein the guard holder is moveable relative to the needle guard extension along the longitudinal axis when the needle guard extension is moved from the engaged position to the active position.

20. The injector of claim 1, wherein the guard holder is prevented from rotating relative to the needle guard extension about the longitudinal axis when the needle guard extension is in the initial position and in the engaged position.

21. An injector comprising:
a housing having a proximal end and a distal end extending along a longitudinal axis extending therebetween;
a needle extending from the distal end of the housing;
a needle shield removably coupled to the needle; and
a guard assembly configured to couple to the distal end of the housing, the guard assembly including:
a guard holder having at least one engagement feature extending radially inward and configured to couple to the needle shield; and
a needle guard extension removably coupled to the guard holder, the needle guard extension (i) coupled to the guard holder and separated from the distal end of the housing in an initial position, (ii) coupled to the guard holder and coupled to the distal end of the housing in an engaged position and (iii) separated from the guard holder and coupled to the distal end of the housing in an active position,
wherein the at least one engagement feature is deformable in response to the needle shield passing thereby when the needle guard extension is moved from the initial position to the engaged position,
wherein the guard holder couples to the needle shield to remove the needle shield from the needle when the needle guard extension is moved from the engaged position to the active position, and
wherein the needle guard extension is retractable relative to the housing between an extended position and a retracted position when the needle guard extension is in the active position.

22. A method of coupling a needle guard to an injector having a housing having a proximal end and a distal end extending along a longitudinal axis extending therebetween, a needle extending from the distal end of the housing, and a needle shield removably coupled to the needle, the method comprising the steps of:
coupling a guard assembly to the distal end of the housing, the guard assembly having a guard holder including at least one engagement feature extending radially inward and configured to couple to the needle shield and a needle guard extension removably coupled to the guard holder and configured to couple to the distal end of the housing;
separating the guard holder from the needle guard extension, thereby removing the needle shield from the needle, where the needle guard extension extends further from the distal end of the housing than the needle; and
pressing the needle guard extension against an injection site of a subject, where the needle guard extension is retractable relative to the housing to expose the needle for delivery of a medicament to the subject.

23. The method of claim 22, wherein the at least one engagement feature deforms in response to the needle shield passing thereby when the guard assembly is coupled to the distal end of the housing.

24. The method of claim 23, wherein the at least one engagement feature includes a plurality of engagement features spaced circumferentially around the guard holder.

25. The method of claim 22, wherein the needle guard extension is threadedly coupled to the distal end of the housing, wherein the needle guard extension includes a knob extending radially therefrom and the housing includes a thread configured to receive the knob.

26. The method of claim 22, wherein the needle extends a predetermined distance past a distal end of the needle guard extension when the needle guard extension is pressed against the injection site of a subject, and wherein the predetermined distance is an insertion depth.
